(19) United States
(12) Patent Application Publication
(10) Pub. No.: US 2008/0305115 A1
(43) Pub. Date: Dec. 11, 2008

(54) Reduced-mass, long-acting dosage forms

(76) Inventors: Thomas R. Tice, Indian Springs, AL (US); Peter Markland, Birmingham, AL (US); Jay K. Staas, Pelham, AL (US)

Correspondence Address: Ballard Spahr Andrews & Ingersoll, LLP, SUITE 1000, 999 PEACHTREE STREET, ATLANTA, GA, 30309-3915, US

(21) Appl. No.: 12/157,368

(22) Filed: Jun. 9, 2008

Related U.S. Application Data
(60) Provisional application No. 60/933,647, filed on Jun. 7, 2007.

(51) Int. Cl. A61K 39/395; A61K 31/7088

(52) U.S. Cl. 424/158.1; 514/44; 424/130.1

(57) ABSTRACT

Methods and compositions are disclosed whereby free antibody or nucleic acid co-administered with a long-acting formulation, such as a microparticle or implant, containing the antibody or nucleic acid to achieve a long duration of antibody or nucleic acid release. One result is that less of the long-acting formulation excipient or polymer is needed allowing for small-volume administrations as required, for example, for ocular, intra-dermal, orthopedic, brain and spinal delivery. In one aspect, the free antibody or nucleic acid alone has efficacy for an extended period, during which time, very little or no long-acting formulation antibody or nucleic acid is released. In one aspect, after the free antibody or nucleic acid has diminished activity, is gone, or no longer has activity, the long-acting formulation antibody or nucleic acid begins to release for a desired preprogrammed duration to provide long-acting durations. Less formulation mass is needed because the entire antibody or nucleic acid is not encapsulated or implanted with encapsulation or implant excipient or polymer. In addition, more antibody or nucleic acid can be administered to afford longer-acting formulations.

This application claims the benefit of and priority to U.S. Provisional Application No. 60/933,647, filed Jun. 7, 2007, which is hereby incorporated herein by reference in its entirety.

### FIELD OF THE INVENTION

The present invention relates to a method of delivering an antibody or a nucleic acid by administration of a free antibody or a free nucleic acid and a long-acting (or sustained-release) pharmaceutical dosage form of the antibody or nucleic acid.

### BACKGROUND

The design and development of long-acting or sustained-release delivery formulations have been the focus of considerable efforts in the pharmaceutical industry for decades. Parenteral formulations and, in particular, those that can be administered by injection or implantation, are particularly useful to achieve systemic and local delivery of bioactive agents for extended periods of times. The benefits of such dosage forms are multifold. Less frequent dosing afforded by long-acting formulations can benefit the patient by simply reducing the number and frequency of times that they need to be administered with the formulation. When administration involves injections or other clinical procedures, then, reducing frequency of injections is a benefit to the patient in terms of reducing patient discomfort, pain, and inconvenience—particularly for injections into the eye or the spinal cord or other sensitive sites. Moreover, the constant delivery of the bioactive agent for long periods of time can also improve compliance to the treatment program and, consequently, can improve recovery or treatment response.

Despite the long duration of certain sustained-release formulations of bioactive agents (e.g., up to 3, 6, or 9 months or longer), certain administrations are problematic in that the total volume of administration must be small. That is, the total delivery volume for certain administration routes is limited, and so, the amount of bioactive agent that is delivered is reduced due to the volume taken up by the polymer wall forming or matrix material and/or excipient of the microparticle or implant. The volume taken up by the microparticle or implant can lead to not enough bioactive agent being delivered and/or the bioactive agent not being delivered over a long enough period of time.

There is a need to address the aforementioned problems and other shortcomings associated with the traditional delivery systems and the traditional methods of delivering certain bioactive agents. These needs and other needs are satisfied by the delivery systems and methods of the present invention.

### SUMMARY OF THE INVENTION

The present invention relates to methods and compositions whereby unencapsulated (“free”) antibody or nucleic acid is co-administered with encapsulated (such as by microparticle or implant) antibody or nucleic acid to achieve a long duration of bioactive release. One result is that less encapsulation excipient is needed allowing for small-volume administrations as required, for example, for ocular, intra-dermal, orthopedic, brain, and spinal delivery. In one aspect, the unencapsulated antibody or nucleic acid alone has efficacy for an extended period, during which time, very little or no encapsulated bioactive is released. In one aspect, after the unencapsulated antibody or nucleic acid has reduced its activity, is gone, or no longer has activity, the encapsulated antibody or nucleic acid begins to release for a desired preprogrammed long acting duration. In summary, less formulation mass is needed because not all of the antibody or nucleic acid is encapsulated with encapsulation excipient. In addition, more antibody or nucleic acid can be administered to afford longer acting formulations. Lastly, the use of the invention can be for systemic or local delivery.

In one aspect, it is desirable to take advantage of the long half-life of certain antibodies or nucleic acids when designing and developing the long-acting formulation. In particular, it is beneficial to administer a free antibody or nucleic acid that has a long duration of action (either in the body or at a local site) at the same time as administering or delivering a long-acting formulation containing that same antibody or nucleic acid. In particular, because duration of action of a freely-administered antibody or nucleic acid can persist for some length of time, it is not necessary for the long-acting formulation to be designed to release the antibody or nucleic acid during this initial period of time of free antibody or nucleic acid duration.

In one aspect, the present invention describes a method for administering free antibody or nucleic acid concomitantly with a delayed-release formulation comprising the antibody or nucleic acid. Preferably, the long-acting formulation releases relatively little of the antibody or nucleic acid after administration so that the predominant source of antibody or nucleic acid that is released after administration is from the portion of the antibody or nucleic acid that was administered as the free agent. In another aspect, the invention describes a controlled release formulation to achieve such an administration.

In one broad aspect, the aspect is directed to a method of extending the release profile of an antibody or nucleic acid in a subject while reducing the total system mass of the polymer material of a biodegradable, long-acting formulation comprising administering to the subject at about the same time a free antibody or nucleic acid and a biodegradable, long-acting formulation containing the antibody or nucleic acid, wherein the free antibody or nucleic acid has a pharmaceutically acceptable bioactivity period of at least a week and wherein the biodegradable, long-acting formulation releases its antibody or nucleic acid to coincide with the diminution of activity of the free antibody or nucleic acid.

In another broad aspect, the aspect is directed to a controlled release formulation comprising a free antibody or nucleic acid and a biodegradable, long-acting formulation containing the antibody or nucleic acid, wherein the free antibody or nucleic acid has a pharmaceutically acceptable bioactivity period of at least a week and wherein the biodegradable, long acting formulation releases its antibody or nucleic acid to coincide with the diminution of activity of the free antibody or nucleic acid

In another broad aspect, the aspect is directed to a method of extending the release profile of an antigen or nucleic acid while reducing the total system mass of the polymer wall forming material of a microparticle comprising administering at about the same time a free antigen or nucleic acid and a microparticle containing the antigen or nucleic acid, wherein the free antigen or nucleic acid has a pharmaceutically acceptable bioactivity period of at least a week and wherein the microparticle releases its antigen or nucleic acid to coincide with the diminution of activity of the free antigen or nucleic acid.

In another broad aspect, the aspect is directed to a controlled release formulation comprising a free antigen or nucleic acid and a microparticle containing the antigen or nucleic acid, wherein the free antigen or nucleic acid has a pharmaceutically acceptable bioactivity period of at least a week and wherein the microparticle releases its antigen or nucleic acid to coincide with the diminution of activity of the free antigen or nucleic acid.

Otherwise, the advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION OF THE INVENTION

The present invention can be understood more readily by reference to the following detailed description, examples, and claims, and their previous and following description. However, before the present compositions, articles, devices, and/or methods are disclosed and described, it is to be understood that this invention is not limited to the specific compositions, articles, devices, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its currently known embodiments. To this end, those skilled in the relevant art will recognize and appreciate that many changes can be made to the various aspects of the invention described herein, while still obtaining the beneficial results of the present invention. It will also be apparent that some of the desired benefits of the present, invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

### Definitions

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like; reference to “an antibody or nucleic acid” includes a single antibody or nucleic acid or a mixture comprising two or more antibodies or nucleic acids and the like; similarly, “a polymer” includes a single polymer or a mixture comprising two or more polymers and the like; and so on.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, a “wt. %” or “weight percent” or “percent by weight” of a component, unless specifically stated to the contrary, refers to the ratio of the weight of the component to the total weight of the composition in which the component is included, expressed as a percentage.

By “contacting” is meant an instance of exposure by close physical contact of at least one substance to another substance.

By “sufficient amount” and “sufficient time” means an amount and time needed to achieve the desired result or results, e.g., dissolve a portion of the polymer.

“Admixture” or “blend” is generally used herein to refer to a physical combination of two or more different components. In the case of polymers, an admixture, or blend, of polymers is a physical blend or combination of two or more different polymers.

“Agent” is used herein to refer generally to compounds that are contained in or on the long-acting formulation. Agent may include an antibody or nucleic acid or an excipient or, more generally, any additive in the long-acting formulation. “Agent” includes a single such compound and is also intended to include a plurality of such compounds.

“Bioactive agent” is used herein to include a compound of interest contained in or on a pharmaceutical formulation or dosage form that is used for pharmaceutical or medicinal purposes to provide some form of therapeutic effect or elicit some type of biologic response or activity. As used herein, bioactive agent typically refers to an antibody or nucleic acid. “Bioactive agent” includes a single such agent and is also intended to include a plurality of bioactive agents including, for example, combinations of two or more bioactive agents.

“Excipient” is used herein to include any other agent or compound that may be contained in a long-acting formulation that is not the antibody or nucleic acid. As such, an excipient should be pharmaceutically or biologically acceptable or relevant (for example, an excipient should generally be non-toxic to the subject). “Excipient” includes a single such compound and is also intended to include a plurality of such compounds.

“Biocompatible” as used herein refers to a material that is generally non-toxic to the recipient and does not possess any significant untoward effects to the subject and, further, that any metabolites or degradation products of the material are non-toxic to the subject.

“Biodegradable” is generally referred to herein generally as a material that will erode to soluble species or that will degrade under physiologic conditions to smaller units or chemical species that are, themselves, non-toxic (biocompatible) to the subject and capable of being metabolized, eliminated, or excreted by the subject.

Terms such as “long-acting”, “sustained-release” or “controlled release” are used generally to describe a formulation, dosage form, device or other type of technologies used, such as, for example, in the art to achieve the prolonged or extended release or bioavailability of an antibody or nucleic acid to a subject; it may refer to technologies that provide prolonged or extended release or bioavailability of an antibody or nucleic acid to the general systemic circulation or a subject or to local sites of action in a subject including (but not limited to) cells, tissues, organs, joints, regions, and the like. Furthermore, these terms may refer to a technology that is used to prolong or extend the release of antibody or nucleic acid from a formulation or dosage form or they may refer to a technology used to extend or prolong the bioavailability or the pharmacokinetics or the duration of action of a antibody or nucleic acid to a subject or they may refer to a technology that is used to extend or prolong the pharmacodynamic effect elicited by a formulation. A “long-acting formulation,” a “sustained release formulation,” or a “controlled release formulation” (and the like) is a pharmaceutical formulation, dosage form, or other technology that is used to provide long-acting release of an antibody or nucleic acid to a subject.

The term “modified bioactive agent” and the like is used herein to refer, generally, to a bioactive agent that has been modified with another entity through either covalent means or by non-covalent means. The term also is used to include prodrug forms of bioactive agents, where the prodrug form could be a polymeric prodrug or non-polymeric prodrug. Modifications conducted using polymers could be carried out with synthetic polymers (such as polyethylene glycol, PEG; polyvinylpyrrolidone, PVP; polyethylene oxide, PEO; propylene oxide, PPO; copolymers thereof; and the like) or biopolymers (such as polysaccharides, proteins, polypeptides, among others) or synthetic or modified biopolymers.

The term “microparticle” is used herein to refer generally to a variety of substantially structures having sizes from about 10 nm to 2000 microns (2 millimeters) and includes microcapsule, microsphere, nanoparticle, nanocapsule, nanosphere as well as particles, in general, that are less than about 2000 microns (2 millimeters).

The terms “microencapsulated” and “encapsulated” are used herein to refer generally to an antibody or nucleic acid that is incorporated into any sort of long-acting formulation or technology regardless of shape or design; therefore, a “microencapsulated” or “encapsulated” antibody or nucleic acid may include antibody or nucleic acid that is incorporated into a particle or a microparticle and the like or it may include antibody or nucleic acid that is incorporated into a solid implant and so on.

“Implant” as used herein is intended to refer generally to a controlled release preformed macroscopic device.

“Needle” is used herein to refer to small-diameter devices that can be used to administer, deliver, inject, or otherwise introduce a long-acting formulation to a subject (either animal or human) for any purposes including medical, clinical, surgical, therapeutic, pharmaceutical, pharmacological, diagnostic, cosmetic, and prophylactic purposes. Examples can include, without being limiting, needles, hypodermic needles, surgical needles, infusion needles, catheters, trocars, cannulas, tubes and tubing used for clinical, surgical, medical, procedural, or medical purposes, and the like.

“Subject” is used herein to refer to any target of administration. The subject can be a vertebrate, for example, a mammal. Thus, the subject can be a human. The term does not denote a particular age or sex. Thus, adult and newborn subjects, as well as fetuses, whether male or female, are intended to be covered. A “patient” refers to a subject afflicted with a disease or disorder and includes human and veterinary subjects.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different polymers and agents are disclosed and discussed, each and every combination and permutation of the polymer and agent are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### Introduction and Discussion

The present invention relates to a method of delivering an antibody or nucleic acid by administration of long-acting (or sustained-release) pharmaceutical formulations. More specifically, the present invention provides a method for administering free antibody or nucleic acid (unencapsulated bioactive) concomitantly with a long-acting (delayed-release followed by sustained release) formulation comprising the antibody or nucleic acid. In this manner, the bolus administration of the free antibody or nucleic acid provides an initial supply of antibody or nucleic acid to the subject, which persists for a length of time based on the pharmacokinetics (half-life and duration of action) of the free antibody or nucleic acid. In various aspects of the present invention, antibodies or nucleic acids are used that have a long duration of action from a bolus administration of the free antibody or nucleic acid lasting, for example, at least one week, at least two weeks, at least three weeks, at least four weeks, at least one month, or at least two months, or longer, wherein the bolus administration is able to provide pharmacokinetic antibody or nucleic acid levels in the bloodstream or in the local site that are sufficient to provide therapeutic effects or an otherwise desirable pharmacodynamic response or effect. In one aspect, such long duration antibodies or nucleic acids last at least a week and up to about a month, and in certain cases up to about three months.

After the duration of action of the bolus administration of an antibody or nucleic acid, the subsequent release of the antibody or nucleic acid from the long-acting formulation provides an additional supply of antibody or nucleic acid to the subject for continued therapeutic treatment. The duration of the long acting or sustained-release formulation can be any period of release capable in the art, for example, up to 3, 4, 5, 6, 7, 8, 9, 10, or 11 months or longer. In one aspect, the total release period can be made longer with the present invention because there is no need for any microparticle release during the period of or substantial period of free antibody or nucleic acid duration.

In one aspect, the long-acting formulation releases or delivers relatively little of the antibody or nucleic acid after it is first administered so that the predominant source of antibody or nucleic acid initially after administration is from the free antibody or nucleic acid that is administered in the bolus dose given concomitantly with the long-acting formulation. In this manner, the bolus administration of the free antibody or nucleic acid provides the initial antibody or nucleic acid to the body or the local site and this portion of antibody or nucleic acid then persists for a length of time based on its particular pharmacokinetics profile (half-life and duration of action).

As used herein, the term “the biodegradable, long-acting formulation releases its antibody or nucleic acid to coincide with the diminution of activity of the free antibody or nucleic acid” includes aspects ranging from where the long-acting formulation releases its agent (1) prior to the free agent beginning to diminish its activity (in anticipation of such a free agent diminution in activity), (2) as the free agent begins to diminish in activity, (3) when the free agent is partially diminished in activity, for example at least 25%, at least 50% or at least 75% diminished, (4) when the free agent is substantially diminished in activity, or (5) when the free agent is completely gone or no longer has activity. In one aspect, the gap between the diminution of the free antibody or nucleic acid and the beginning of a sufficient release of the antibody or nucleic acid from the long-acting formulation is minimized. That is, in this aspect, it is undesirable for there to be a period between the free antibody or nucleic acid and long-acting formulation releases of no or low administration, such that, the antibody or nucleic acid is below pharmaceutically acceptable levels in the body. In a further aspect, there is at least an overlap or even a substantial overlap between the period of release of the free antibody or nucleic acid and the period of release from the long-acting formulation to ensure minimum acceptable levels of the active agent in the body are maintained. In another aspect, right after or shortly after the unencapsulated antibody or nucleic acid is gone or no longer has activity, the encapsulated antibody or nucleic acid begins to release for a desired preprogrammed long acting duration.

In one aspect, the present invention describes a method in which the concomitant administration of the bolus dose of free antibody or nucleic acid, along with the long-acting formulation is intended to provide for systemic delivery of the antibody or nucleic acid to the general circulation; or, instead, for local delivery to a local site, tissue, organ or the like; or for combinations thereof. In another regard, the present invention involves a long-acting formulation that is comprised of a non-degradable biocompatible polymer or a degradable biocompatible polymer or of combinations thereof. In one regard, the present invention involves the use of any type of long-acting dosage form including, but not limited to, particles (including microparticles, microspheres, microcapsules, nanoparticles, nanospheres, nanocapsules, and the like) or implants (including injectable implants and those that can be administered in surgical or clinical settings; implants can include solid, semisolid, hydrogel, viscous, liquid implants or combinations thereof and may include implants that transition from one physical form to the other before, during, or after administration). The long acting dosage form can be in the form of an injectable liquid, gel, solution, or suspension.

Local delivery of an antibody or nucleic acid to locations such as organs, cells or tissues can also result in a therapeutically useful, long-lasting presence of antibody or nucleic acid, in those local sites or tissues since the routes by which an antibody or nucleic acid are distributed, metabolized, and eliminated from these locations may be different than the routes that define the pharmacokinetic duration of an antibody or nucleic acid, delivered to the general systemic circulation. The present invention can deliver to any variety of sites, locations, organs, cells, or tissues throughout the body. In one aspect, the delivery is to locations that historically are limited in the volume of administered formulation, that is, only a small amount of formulation volume is capable of being administered. This aspect includes, but is not limited to, a local delivery, an interarticular delivery, such as between the joints, orthopedic sites (bones, bone defects, joints, and the like), CNS locations (including, for example, spinal, cerebrospinal or intrathecal delivery or delivery into the brain or to specific sites in and around the brain), intradermal, intratumor, peritumor, or ocular delivery (to sites adjacent to or ori-the eye, sites within ocular tissue, or intravitreal delivery inside the eye).

In a specific aspect, the invention is directed to delivery to a cancer or to the vasculature associated with a cancer (e.g., to inhibit angiogenesis). The cancer can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. In some aspects, the cancer is a solid tumor. In some aspects, the cancer is carcinoma. In some aspects, the cancer is a sarcoma. In some aspects, the cancer is a lymphoma. In some aspects, the cancer is germ-cell tumor. In some aspects, the cancer is blastic tumor. A representative but non-limiting list of cancers include B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

In a specific aspect, the invention is directed to delivery to ocular delivery. Typically, free antibody or nucleic acid used to treat maladies of the eye last only up to two months and the total volume of administrated formulation is limited to up to about 50 μL or up to about 100 μL in certain cases. With the present invention, by using a free antibody or nucleic acid and a long-acting formulation, the volume limitations can still be met while producing a pharmaceutical effect of over two months. For example, compositions of the invention can be administered as a single injection into the eye to achieve efficacy that lasts for 3, 6, or 9 months or longer eliminating up to 3 injections into the eye.

With respect to the term, “administering at about the same time,” as used herein, the method of the present invention may be practiced in a variety of ways including, but not limited to, delivering the free agent at the same time as the long-acting formulation or delivering the free agent sequentially to (either before or after) the delivery of the long-acting formulation. The time period between deliveries is intended to be reasonably short. Such administering at about the same time, includes, but is not limited to, the following: the combined administration of the free antibody or nucleic acid and the long-acting formulation in the same surgical or clinical procedure (for example, the co-administration of both in the same injection or during the same surgical intervention); or, in separate procedures that may be performed one after the other (as in the case of the injection of the bolus dose of the free antibody or nucleic acid in one procedure followed by (or preceded by) the administration of the long-acting formulation in a second procedure, for example, by the injection of a bolus dose of the free antibody or nucleic acid followed by a second injection of the long-acting formulation); or, concomitantly (at the same time) but in different procedures (as in the case of an infusion administration of the bolus free antibody or nucleic acid during which time the long-acting formulation is administered by, for example, injection or implantation). When the free antibody or nucleic acid and the long-acting formulation are performed in the same procedure, for example, in the same injection, the free antibody or nucleic acid can be combined with the long-acting formulation in a simple admixture. For example, the free antibody or nucleic acid can be dissolved or suspended in a solvent such as water and the microencapsulated formulation can be suspended in the same water solution.

In one aspect, the administration is to a subject in need of such administering.

### Long-Acting Formulations

The method of the present invention includes the use of any type of long-acting formulation or dosage form that may be used (or envisioned to be used) for delivery of an antibody or nucleic acid to prolong or extend an antibody or nucleic acid, such as a antibody or nucleic acid release, bioavailability, pharmacokinetics, pharmacodynamic effects or profiles.

Generally, long-acting or sustained release formulations comprise an agent or agents (including, for example, an antibody or nucleic acid) that is/are incorporated or associated with a biocompatible polymer in one manner or another. The matrix-forming polymers typically used in the preparation of long-acting formulations include, but are not limited, to biodegradable polymers (such as the polyesters poly(lactide), poly(lactide-co-glycolide), poly(caprolactone), poly(hydroxybutyrates), and the like) and non-degradable polymers (such as ethylenevinyl acetate (EVA), silicone polymers, and the like). The agent may be blended homogeneously throughout the polymer matrix or the agent may be distributed unevenly (or discontinuously or heterogeneously) throughout the polymer matrix (as in the case of an antibody or nucleic acid-loaded core that is surrounded by a polymer-rich coating or polymer wall forming material as in the case of a microcapsule, nanocapsule, a coated or encapsulated implant, and the like). The dosage form may be in the physical form of particles, film, a fiber, a filament, a cylindrical implant, a asymmetrically-shaped implant, or a fibrous mesh (such as a woven or non-woven material; felt; gauze, sponge, and the like). When in the form of particles, the formulation may be in the form of microparticles, nanoparticles, microspheres, nanospheres, microcapsules or nanocapsules, and particles, in general, and combinations thereof. As such, the long-acting (or sustained-release) formulations of the present invention may include any variety of types or designs that are described, used or practiced in the art.

Long-acting formulations containing an antibody or nucleic acid can be used to deliver those agents to the systemic circulation or they can be used to achieve local or site-specific delivery to cells, tissues, organs, bones and the like that are located nearby the site of administration. Further, formulations can be used to achieve systemic delivery of the antibody or nucleic acid and/or local delivery of the antibody or nucleic acid. Formulations can be delivered by injection (through, for example, needles, syringes, trocars, cannula, and the like) or by implantation. Delivery can be made via any variety of routes of administration commonly used for medical, clinical, surgical purposes including, but not limited to, intravenous, intraarterial, intramuscular, intraperitoneal, subcutaneous, intradermal, infusion and intracatheter delivery (and the like) in addition to delivery to specific locations (such as local delivery) including intrathecal, intracardiac, intraosseous (bone marrow), stereotactic-guided delivery, infusion delivery, CNS delivery, stereo-tactically administered delivery, orthopedic delivery (for example, delivery to joints, into bone, into bone defects and the like), cardiovascular delivery, inter- and intra- and para-ocular (including intravitreal and scleral and retrobulbar and sub-tenons delivery and the like), any delivery to any multitude of other sites, locations, organs, tissues, etc.

In one aspect, the method of the present invention therefore envisions utilizing any technology that is used (or may be envisioned to be used) in the field for parenteral routes of administration including, for example but without being limited to those described by: Maindares and Silva, Curr Drug Targets, 5(5), 449 (2004); or, Degim and Celebi, Curr Pharm Des, 13(1), 99 (2007); or, Encyclopedia of Pharmaceutical Technology, James Swarbrick and James Boylan (Editors), Marcel Dekker, New York (2004); or, Encyclopedia of Controlled Drug Delivery, Edith Mathiowitz (Editor); John Wiley & Sons, New York (1999); or Controlled Release Veterinary Drug Delivery, Robert Gurny and Michael J. Rathbone (Editors); Elsevier Science B. V., Amsterdam, The Netherlands (2000); or Encyclopedia of Nanoscience and Nanotechnology, James Schwarz, Cristian Contescu, Karol Putyera (Editors), Marcel Dekker, Inc., New York (2004); or Encyclopedia of Biomaterials and Biomedical Engineering, Gary Wnek and Gary Bowlin (Editors), Marcel Dekker, Inc., New York (2004); or, Malik, Baboota, Ahuja, and Hassan, Curr Drug Deliv., 4(2), 141 (2007); or Nair and Laurencin, Adv Biochem Eng Biotechnol, 102, 47 (2006); and the like. All of the above references are incorporated herein by this reference for all of their teachings as well as for the specific teachings of parenteral route technology methods.

In one aspect, the method of the present invention includes long-acting formulations that can be administered by needle, injection, infusion, implantation (as might be conducted either clinically or surgically), and the like.

### Polymers and Excipients

Polymers used to prepare the long-acting formulation can be any biocompatible polymer. One of skill in the art would know how to select without undue experimentation the proper polymer composition to achieve the desired effect of, in one aspect, allowing the free antibody or nucleic acid to provide its effect, and then, staging in the release of the antibody or nucleic acid from the long-acting formulation at an appropriate time about on or after the free antibody or nucleic acid provides its effect, as described above. In one aspect the polymer is selected to delay the release of the antibody or nucleic acid until some time after the free agent has provided its effect, thereby extending the total effect period. Such selection of the polymer can include criteria, such as, for example, the type of polymer, the selection of a polymer or a co-polymer, the type of co-monomers used in the co-polymer, the ratio of the types of monomers used in the co-polymer, the molecular weight of the polymer, the size of the microparticle, and any other criteria that is used by one of skill in the art to control the release profile of a microparticle.

Without intending to be limiting, examples may include any biocompatible polymers used in the art. For example, biocompatible non-degradable polymers can be used including, for example, a polyacrylate; a polymer of ethylene-vinyl acetate, EVA; cellulose acetate; an acyl-substituted cellulose acetate; a non-degradable polyurethane; a polystyrene; a polyvinyl chloride; a polyvinyl fluoride; a poly(vinyl imidazole); a silicone-based polymer (for example, Silastic® and the like), a chlorosulphonate polyolefin; a polyethylene oxide; or a blend or copolymer thereof. Biocompatible biodegradable polymers can be used including, but not limited to, a poly(lactide); a poly(glycolide); a poly(lactide-co-glycolide); a poly(lactic acid); a poly(glycolic acid); a poly(lactic acid-co-glycolic acid); a poly(caprolactone); a poly(orthoester); a polyanhydride; a poly(phosphazene); a polyhydroxyalkanoate; a poly(hydroxybutyrate); a poly(hydroxybutyrate) synthetically derived; a poly(hydroxybutyrate) biologically derived; a polyester synthetically derived; a polyester biologically derived; a poly(lactide-co-caprolactone); a poly(lactide-co-glycolide-co-caprolactone); a polycarbonate; a tyrosine polycarbonate; a polyamide (including synthetic and natural polyamides, polypeptides, poly(amino acids) and the like); a polyesteramide; a polyester; a poly(dioxanone); a poly(alkylene alkylate); a polyether (such as polyethylene glycol, PEG, and polyethylene oxide, PEO); polyvinyl pyrrolidone or PVP; a polyurethane; a polyetherester; a polyacetal; a polycyanoacrylate; a poly(oxyethylene)/poly(oxypropylene) copolymer; a polyacetal, a polyketal; a polyphosphate; a (phosphorous-containing) polymer; a polyphosphoester; a polyhydroxyvalerate; a polyalkylene oxalate; a polyalkylene succinate; a poly(maleic acid); biopolymers or modified biopolymers including chitin, chitosan, modified chitosan, among other biocompatible polysaccharides; or biocompatible copolymers (including block copolymers or random copolymers) herein; or combinations or mixtures or admixtures of any polymers herein. Examples of copolymers that could be used include block copolymers containing blocks of hydrophilic or water-soluble polymers (such as polyethylene glycol, PEG, or polyvinyl pyrrolidone, PVP) with blocks of other biocompatible or biodegradable polymers (for example, poly(lactide) or poly(lactide-co-glycolide or polycaprolcatone or combinations thereof).

Furthermore, the present invention also relates to long-acting formulations prepared from copolymers that are comprised of the monomers of lactide (including L-lactide, D-lactide, and combinations thereof) or hydroxybutyrates or caprolactone or combinations thereof; and to long-acting formulations prepared from copolymers that are comprised of the monomers of DL-lactide, glycolide, hydroxybutyrate, and caprolactone and to long-acting formulations prepared from copolymers comprised of the monomers of DL-lactide or glycolide or caprolactone or hydroxybutyrates or combinations therein. Additionally, the present invention also relates to long-acting formulations prepared from admixtures containing the aforementioned copolymers (comprised of DL-lactide or glycolide or caprolactone or hydroxybutyrates or combinations therein) along with other biodegradable polymers including poly(DL-lactide-co-glycolide) or poly(DL-lactide) or PHA's, among others. The present invention can further include long-acting formulations prepared from block copolymers comprised with blocks of either hydrophobic or hydrophilic biocompatible polymers or biopolymers or biodegradable polymers such as polyethers (including polyethylene glycol, PEG; polyethylene oxide, PEO; polypropylene oxide, PPO and block copolymers comprised of combinations thereof) or polyvinyl pyrrolidone (PVP), polysaccharides, conjugated polysaccharides, modified polysaccharides, such as fatty acid conjugated polysaccharides, polylactides, polyesters, among others.

With the practice of the aspects herein, such as the combination of a delivery of the free antibody and nucleic acid along with the delivery of a long-acting formulation of the free antibody and nucleic acid, the polymer material (and in some aspects the excipient material) system mass is reduced due to less antibody or nucleic acid needed in the long-acting formulation.

### Free Administration

The administration of the free antibody or nucleic acid (i.e., unencapsulated) is performed using any method known in the art, such as by injection or infusion. One of skill in the art readily knows how to prepare a formulation of and administer a free antibody or nucleic acid. As discussed above, the free antibody or nucleic acid can be delivered in the same formulation as the long-acting formulation in one unitary formulation or the free antibody or nucleic acid can be delivered separately from the long-acting formulation. In one aspect, the free antibody or nucleic acid is delivered in the same formulation as the long-acting formulation as one unitary formulation.

The antibody or nucleic acid used in the free administration is typically the same or essentially the same as the antibody or nucleic acid used in the long-acting formulation. In one aspect the antibody or nucleic acid is the same as that used in the long-acting formulation.

### Bioactive Agents

Any antibody or nucleic acid for the free antibody or free nucleic acid and the long-acting formulation can be utilized. Some antibodies or nucleic acids have long lasting effects (days or weeks) in the body or in local tissues (when administered for local delivery) after administration, such as antibodies or nucleic acids that have half lives ranging from about 1.7 to 12 days or as long as 20 to 50 days or longer than 50 days. Modified antibodies or nucleic acids can also exhibit prolonged or extended pharmacokinetic profiles, for example, achieving a plasma half-life of about 3-6 days to as long as 14 days or 1 month or 2 months or 3 months or longer. One aspect of the present invention involves the use of an antibody or nucleic acid that can provide a duration of action, i.e., a pharmaceutically acceptable bioactivity period (therapeutically efficacious blood or tissue concentrations over time) that extends for at least one week, at least two weeks, at least three weeks, at least four weeks, at least one month, at least two months, or at least three months or longer following a single bolus administration. In another aspect, any antibody or nucleic acid can be used in the present invention.

In one aspect, the antibody or nucleic acid is an antibody. In a specific aspect, the antibody is a therapeutic antibody. In another aspect, the antibody is a therapeutic antibody fragment. In another aspect, the antibody is a nanobody. In another aspect, the antibody is an Fab fragment. In another aspect, the antibody is an F(ab)₂ fragment. In another aspect, the antibody is a single chain antibody. In another aspect, the antibody is a chimeric antibody. In another aspect, the antibody is a humanized antibody. In another aspect, the antibody is a recombinant antibody. In another aspect, the antibody is a human antibody. In another aspect, the antibody is a monoclonal antibody. In another aspect, the antibody is a polyclonal antibody.

In still another aspect, the antibody or nucleic acid is a nucleic acid. In yet another aspect, the nucleic acid is an aptamer, iRNA, siRNA, DNA, RNA, antisense nucleic acid or the like, or an antisense nucleic acid analog or the like. In yet another aspect, the nucleic acid is a small interfering RNA (siRNA). In yet another aspect, the nucleic acid is an antisense oligonucleotide. In yet another aspect, the nucleic acid is a nucleic acid encoding a protein. In yet another aspect, the nucleic acid is a vector comprising a nucleic acid encoding a protein operably linked to an expression control sequence.

In yet another aspect, the antibody or nucleic acid is a modified antibody or nucleic acid. In one aspect, the antibody is a PEG-modified antibody. In another aspect, the antibody is a PEG-modified antibody fragment. In still another aspect, the nucleic acid is a PEG-modified nucleic acid.

The antibody or nucleic acid is used for the treatment, diagnosis, cure or mitigation of disease or illness, a substance which affects the structure or function of the body, or pro-drugs, which become biologically active or more active after they have been placed in a predetermined physiological environment. In a specific aspect, the antibody or nucleic acid is not a vaccine. Antibodies or nucleic acids include biologically, physiologically, or pharmacologically active substances that act locally or systemically in the human or animal body. Various forms of the antibodies or nucleic acids can be used, which are capable of being released from the solid matrix into adjacent tissues or fluids. A liquid or solid antibody or nucleic acid can be incorporated in the delivery systems described herein. The antibodies or nucleic acids are at least very slightly water soluble, preferably moderately water soluble, and are diffusible through the polymeric composition. They can be acidic, basic, or amphoteric salts. They can be in the free acid or free base form. They can be nonionic molecules, polar molecules, or molecular complexes capable of hydrogen bonding. The antibody or nucleic acid may be included in the compositions in the form of, for example, an uncharged molecule, a molecular complex, a salt, an ether, an ester, an amide, polymer-drug conjugate, or other form to provide the effective biological or physiological activity.

The long-acting formulations of the present invention can comprise one antibody or nucleic acid or combinations of two or more antibodies or nucleic acids including a large number of antibodies or nucleic acids. The antibody or nucleic acid can be naturally-occurring, produced from fermentation or bacterial sources, or synthetic in origin or they can be prepared from a combination therein. The antibody or nucleic acid can be a compound that has been covalently or non-covalently modified using other materials. Examples include salt counter-ions, targeting agents, solubility modifiers, permeability modifiers, hydrophobic agents, hydrophilic agents, hydrophobic polymers, hydrophilic polymers, block copolymers, and the like.

The antibody of the disclosed compositions and methods can be an antibody, such as a therapeutic antibody. The term “antibody” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with an antigen such that the antigen is inhibited from interacting with its target, such as a ligand or receptor. The antibodies can be tested for their desired activity using the in vitro assays described herein, or by analogous methods, after which their in vivo therapeutic and/or prophylactic activities are tested according to known clinical testing methods.

As used herein, therapeutic antibodies are antibodies that are administered to a subject based on the ability of the antibody to bind a target antigen. They are therefore distinct from vaccines which are administered to a subject to induce an immune response in the subject thereby generating endogenous antibodies to the antigen. Therapeutic antibodies are known in the art and continue to be identified. The herein disclosed methods can be used with any antibody discovered to have a therapeutic effect when it binds its antigen. The antigen can be on a cell, such as a cancer cell. The antigen can be a growth factor. The antigen can be an extracellular structural protein.

The antigen of the disclosed antibody can be, for example, tumour necrosis factor alpha (TNFα). Infliximab (REMICADE) is a chimeric monoclonal antibody (murine binding VK and VH domains and human constant Fc domains) used to treat autoimmune disorders. Infliximab binds TNFα thereby preventing it from signaling its receptors on the surface of cells. TNFα is one of the key cytokines that triggers and sustains the inflammation response. Infliximab has been approved by the U.S. Food and Drug Administration for the treatment of psoriasis, Crohn's disease, ankylosing spondylitis, psoriatic arthritis, rheumatoid arthritis, and ulcerative colitis.

The antigen of the disclosed antibody can be, for example, a vascular endothelial growth factor (VEGF), such as VEGF-A. VEGF is involved in the growth of new blood vessels and vascular permeability. As such, inhibition of its activity is useful for treating or preventing leaky blood vessels or tumor angiogenesis.

In a specific aspect, the antibody is VEGF-trap (AFLIBERCEPT). VEGF-trap is a protein comprised of segments of the extracellular domains of human vascular endothelial growth factor receptors 1 (VEGFR1) and 2 (VEGFR2) fused to the constant region (Fc) of human IgG1 with potential antiangiogenic activity. Afilbercept, functioning as a soluble decoy receptor, binds to pro-angiogenic vascular endothelial growth factors (VEGFs), thereby preventing VEGFs from binding to their cell receptors. Disruption of the binding of VEGFs to their cell receptors may result in the inhibition of tumor angiogenesis, metastasis, and ultimately tumor regression.

Bevacizumab (trade name Avastin) is a monoclonal antibody against VEGF. It is used in the treatment of cancer, where it inhibits tumor growth by blocking the formation of new blood vessels. Bevacizumab can be used in combination with standard chemotherapy in the treatment of metastatic colon cancer and most forms of metastatic non-small cell lung cancer. Bevacizumab can be used at least to treat in breast cancer, metastatic renal cell carcinoma, metastatic glioblastoma multiforme, metastatic ovarian cancer, metastatic hormone-refractory prostate cancer, and metastatic or unresectable locally advanced pancreatic cancer.

Ranibizumab (LUCENTIS) is a monoclonal antibody fragment derived from the same parent murine antibody as bevacizumab (AVASTIN). It is much smaller than the parent molecule and has been affinity matured to provide stronger binding to VEGF-A. It has been approved to treat the “wet” type of age-related macular degeneration (ARMD), a common form of age-related vision loss. Ranibizumab binds to and inhibits all subtypes of vascular endothelial growth factor A (VEGF-A). VEGF can trigger the growth of new vessels, which can leak blood and fluid into the eye. These leaky blood vessels can contribute to macular edema and choroidal neovascularization, resulting in the wet type of ARMD. By blocking VEGF-A in the eye, ranibizumab can prevent and reverse vision loss caused by wet macular degeneration.

The antigen of the disclosed antibody can be, for example, cluster of differentiation 20 (CD20). CD20 is widely expressed on B-cells. Rituximab (RITUXAN, MABTHERA) is a chimeric monoclonal antibody used in the treatment of B cell non-Hodgkin's lymphoma, B cell leukemia, and some autoimmune disorders. Rituximab depletes B cells, and therefore is used to treat diseases which are characterized by having too many B cells, overactive B cells or dysfunctional B cells. Rituximab can be used to treat rheumatoid arthritis and autoimmune diseases, including idiopathic autoimmune hemolytic anemia, Pure red cell aplasia, idiopathic thrombocytopenic purpura (ITP), Evans syndrome, vasculitis, multiple sclerosis, bullous skin disorders (for example pemphigus, pemphigoid), type 1 diabetes mellitus, Sjogren's syndrome, Devic's Syndrome and systemic lupus erythematosus. Rituximab can also be used in the management of Renal Transplant recipients.

Ibritumomab tiuxetan (ZEVALIN) is a monoclonal antibody that binds to the CD20 antigen found on the surface of normal and malignant B cells (but not B cell precursors), allowing radiation from the attached isotope (mostly beta emission) to kill it and some nearby cells. This antibody can be used in radioimmunotherapy treatment for some forms of B cell non-Hodgkin's lymphoma. The drug uses the monoclonal mouse IgG1 antibody ibritumomab in conjunction with the chelator tiuxetan, to which a radioactive isotope (either yttrium-90 or indium-111) is added. In addition, the antibody itself can trigger cell death via antibody-dependent cell-mediated cytotoxicity (ADCC), complement-dependent cytotoxicity (CDC), and apoptosis. Together, these actions eliminate B cells from the body, allowing a new population of healthy B cells to develop from lymphoid stem cells.

The antigen of the disclosed antibody can be, for example, beta-amyloids. The pathology of Alzheimer's disease shows a significant correlation between beta-amyloid peptide conformation and the clinical severity of dementia. Site-directed antibodies can modulate formation of beta-amyloid. Moreover, the antibodies can dissolve β-amyloid plaques and protect the subject from learning and age-related memory deficits.

The antigen of the disclosed antibody can be, for example, α4-integrin. Natalizumab is a humanized monoclonal antibody against the cellular adhesion molecule α4-integrin. Natalizumab is used in the treatment of multiple sclerosis and Crohn's disease. Natalizumab has prevents relapse, vision loss, cognitive decline and significantly improving quality of life in people with multiple sclerosis, as well as increasing rates of remission and preventing relapse in Crohn's disease.

The antigen of the disclosed antibody can be, for example, HER2/neu. Trastuzumab (HERCEPTIN) is a humanized monoclonal antibody that acts on the HER2/neu (erbB2) receptor. Trastuzumab's principal use is as an anti-cancer therapy in breast cancer in patients whose tumors over express (produce more than the usual amount of) this receptor. Amplification of HER2/neu (ErbB2) occurs in 20-30% of early-stage breast cancers. It encodes the extracellular domain of HER2.

The antigen of the disclosed antibody can be, for example, epidermal growth factor receptor (EGFR). Cetuximab (ERBITUX) is a chimeric monoclonal antibody specific for EGFR given by intravenous injection for treatment of metastatic colorectal cancer and head and neck cancer. Panitumumab (VECTIBIX) is another EGFR antibody being used. One of the main differences is that Cetuximab is an IgG1 antibody, and Panitumumab an IgG2 antibody. Cetuximab is binds the extracellular domain of the EGFR of all cells that express EGFR, which includes the subset “cancer cells”, preventing ligand binding and activation of the receptor: This blocks the downstream signaling of EGFR resulting in impaired cell growth and proliferation. Cetuximab has also been shown to mediate antibody dependent cellular cytotoxicity (ADCC).

The antigen of the disclosed antibody can be, for example, CD52. Alemtuzumab (CAMPATH, MABCAMPATH, or CAMPATH-1H) is a monoclonal antibody that targets CD52, a protein present on the surface of mature lymphocytes, but not on the stem cells from which these lymphocytes were derived. It is used in the treatment of chronic lymphocytic leukemia (CLL) and T-cell lymphoma. Alemtuzumab is also used in some conditioning regimens for bone marrow transplantation and kidney transplantation. It can also be used for treatment of autoimmune diseases, such as multiple sclerosis.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

If these approaches do not produce neutralizing antibodies, cells expressing cell surface localized versions of these proteins will be used to immunize mice, rats or other species. Traditionally, the generation of monoclonal antibodies has depended on the availability of purified protein or peptides for use as the immunogen., More recently DNA based immunizations have shown promise as a way to elicit strong immune responses and generate monoclonal antibodies. In this approach, DNA-based immunization can be used, wherein DNA encoding extracellular fragments of the antigen expressed as a fusion protein with human IgG1 or an epitope tag is injected into the host animal according to methods known in the art (e.g., Kilpatrick K E, et al. Gene gun delivered DNA-based immunizations mediate rapid production of murine monoclonal antibodies to the Flt-3 receptor. Hybridoma. 1998 December; 17(6):569-76; Kilpatrick K E et al. High-affinity monoclonal antibodies to PED/PEA-15 generated using 5 microg of DNA. Hybridoma. 2000 August; 19(4):297-302, which are incorporated herein by referenced in full for the methods of antibody production) and as described in the examples.

An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing the extracellular domain of the antigen as fusion proteins with a signal sequence fragment. The antigen is produced by inserting a gene fragment in-frame between the signal sequence and the mature protein domain of the antigen's nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab or F(ab)₂ fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields an Fc fragment and an F(ab)₂ fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

i. Whole Immunoglobulin

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effect or functions, such as participation of the antibody in antibody-dependent cellular toxicity.

ii. Antibody Fragments

The term “antibody” as used herein is meant to include intact molecules as well as fragments thereof, such as, for example, Fab and F(ab′)₂, which are capable of binding the epitopic determinant.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain antigen binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

Also disclosed are fragments of antibodies which have bioactivity. The polypeptide fragments can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with antigen. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypepuide. Such methods are readily apparent to a skilled practitioner in the art and can include site-specific mutagenesis of the nucleic acid encoding the antigen. (Zoller M J et al. Nucl. Acids Res. 10:6487-500 (1982).

Techniques can also be adapted for the production of single-chain antibodies specific to an antigenic protein of the present disclosure (see e.g., U.S. Pat. No. 4,946,778). In addition, methods can be adapted for the construction of F (ab) expression libraries (see e.g., Huse, et al., 1989 Science 246: 1275-1281) to allow rapid and effective identification of monoclonal F (ab)fragments with the desired specificity for a protein or derivatives, fragments, analogs or homologs thereof. Antibody fragments that contain the idiotypes to a protein antigen may be produced by techniques known in the art including, but not limited to: (i) an F ((ab′))(2)fragment produced by pepsin digestion of an antibody molecule; (ii) an Fab fragment generated by reducing the disulfide bridges of an F ((ab′))(2)fragment; (iii) an F (ab)fragment generated by the treatment of the antibody molecule with papain and a reducing agent and (iv) F (v), fragments.

Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding (Bedzyk et al., 1990; Chaudhary et al., 1990). The linker is chosen to permit the heavy chain and light chain to bind together in their proper conformational orientation. See, for example, Huston, J. S., et al., Methods in Enzym. 203:46-121 (1991), which is incorporated herein by reference. These Fvs lack the constant regions (Fc) present in the heavy and light chains of the native antibody.

iii. Monovalent Antibodies

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

iv. Chimeric/Hybrid

In hybrid antibodies, one heavy and light chain pair is homologous to that found in an antibody raised against one antigen recognition feature, e.g., epitope, while the other heavy and light chain pair is homologous to a pair found in an antibody raised against another epitope. This results in the property of multi-functional valency, i.e., ability to bind at least two different epitopes simultaneously. As used herein, the term “hybrid antibody” refers to an antibody wherein each chain is separately homologous with reference to a mammalian antibody chain, but the combination represents a novel assembly so that two different antigens are recognized by the antibody. Such hybrids can be formed by fusion of hybridomas producing the respective component antibodies, or by recombinant techniques. Such hybrids may, of course, also be formed using chimeric chains.

v. Anti-Idiotypic

The encoded antibodies can be anti-idiotypic antibodies (antibodies that bind other antibodies) as described, for example, in U.S. Pat. No. 4,699,880. Such anti-idiotypic antibodies could bind endogenous or foreign antibodies in a treated individual, thereby to ameliorate or prevent pathological conditions associated with an immune response, e.g., in the context of an autoimmune disease.

vi. Conjugates or Fusions of Antibody Fragments

The targeting function of the antibody can be used therapeutically by coupling the antibody or a fragment thereof with a therapeutic agent. Such coupling of the antibody or fragment (e.g., at least a portion of an immunoglobulin constant region (Fc)) with the therapeutic agent can be achieved by making an immunoconjugate or by making a fusion protein, comprising the antibody or antibody fragment and the therapeutic agent.

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference. In some aspects, the antibody does not comprise an immunoglobulin variable region but instead is a fusion protein comprising an immunogolublin Fc region and a binding region of a ligand or receptors.

An antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e. g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates disclosed can be used for modifying a given biological response. The drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, [agr]-interferon, [bgr]-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

vii. Method of Making Antibodies Using Protein Chemistry

One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant Ga. (1992) Synthetic Peptides: A User Guide. W.H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

viii. Human and Humanized

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R.: Liss, p. 77 (1985); Boerner et al., J. Immunol., 147(1):86-95 (1991)).

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementarity determining region (CDR) of the recipient antibody are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992))

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, a humanized form of a non-human antibody (or a fragment thereof) is a chimeric antibody or fragment (U.S. Pat. No. 4,816,567), wherein substantially less than an intact human variable domain, has been substituted by the corresponding sequence from-a non-human species. In practice, humanized-antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

As used herein, the term; “epitope” is meant to include any determinant capable of specific interaction with the antibodies disclosed. Epitopic determinants usually consist of chemically active surface groupings of molecules such as amino acids or sugar side chains and usually have specific three dimensional structural characteristics, as well as specific charge characteristics.

An “epitope tag” denotes a short peptide sequence unrelated to the function of the antibody or molecule that can be used for purification or crosslinking of the molecule with anti-epitope tag antibodies or other reagents.

By “specifically binds” is meant that an antibody recognizes and physically interacts with its cognate antigen and does not significantly recognize and interact with other antigens; such an antibody may be a polyclonal antibody or a monoclonal antibody, which are generated by techniques that are well known in the art.

The antibody can be bound to a substrate or labeled with a detectable moiety or both bound and labeled. The detectable moieties contemplated with the present compositions include fluorescent, enzymatic and radioactive markers.

ix. Administration of Antibodies

Administration of the antibodies can be done as disclosed herein. Nucleic acid approaches for antibody delivery also exist. The broadly neutralizing antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

The nucleic acid of the disclosed compositions and methods can be any nucleic acid. For example, the nucleic acid can be a functional nucleic acid or a nucleic acid encoding a therapeutic protein or peptide. Thus, the disclosed composition can comprise a vector comprising a nucleic acid, wherein the nucleic acid is a functional nucleic acid or a nucleic acid that encodes a therapeutic protein or peptide. The disclosed nucleic acids can be made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

i. Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by-finding the most accessible regions of the target molecule exist. Exemplary methods would be in vitro selection experiments and DNA modification studies using DMS and DEPC. It is preferred that antisense molecules bind the target molecule with a dissociation constant (K_{d})less than or equal to 10⁻⁶, 10⁻⁸, 10⁻¹⁰, or 10⁻¹². A representative sample of methods and techniques which aid in the design and use of antisense molecules can be found in U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptarners can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with K_{d}'s from the target molecule of less than 10-12 M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule (U.S. Pat. No. 5,543,293). It is preferred that the aptamer have a K_{d} with the target molecule at least 10, 100, 1000, 10,000, or 100,000 fold lower than the K_{d} with a background binding molecule. It is preferred when doing the comparison for a polypeptide for example, that the background molecule be a different polypeptide. Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a -number of different types of ribozymes that catalyze nuclease or nucleic acid polymerase type reactions which are based on ribozymes found in natural systems, such as hammerhead ribozymes, (U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203; International Patent Application Nos. WO 9858058 by Ludwig and Sproat, WO 9858057 by Ludwig and Sproat, and WO 9718312 by Ludwig and Sproat) hairpin ribozymes (for example, U.S. Pat. Nos. 5,631,115, 5,646,031, 5,683,902, 5,712,384, 5,856,188, 5,866,701, 5,869,339, and 6,022,962), and tetrahymena ribozymes (for example, U.S. Pat. Nos. 5,595,873 and 5,652,107). There are also a number of ribozymes that are not found in natural systems, but which have been engineered to catalyze specific reactions de novo (for example, U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹². Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). This silencing was originally observed with the addition of double stranded RNA (dsRNA) (Fire,A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III—like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism. p Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. Disclosed herein are any shRNA designed as described above based on the sequences for the herein disclosed inflammatory mediators.

ii. Nucleic Acid Vector

There are a number of compositions and methods which can be used to deliver nucleic acids, such as nucleic acids encoding therapeutic proteins and peptides, to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods can be modified to specifically function with large DNA molecules. Further, these methods can be used to target certain diseases and cell populations by using the targeting characteristics of the carrier.

a. Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport nucleic acids into a cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (A) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### (B) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### (C) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (D) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

b. Non-Nucleic Acid Based Systems

The nucleic acid of the disclosed compositions and methods can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue, the principles of which can be applied to targeting of other cells (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug; targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has, enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (A) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banedji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### (B) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR—cells and mouse LTK—cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

Other materials, including therapeutic, bioactive, diagnostic, and/or prophylactic agents, cells or whole tissues, may be included in the long-acting formulations used in the present invention. These materials can be used, for example, for controlled release of a drug, to render the devices radio-opaque, stimulate tissue in-growth, promote tissue regeneration, prevent infection, or modify the porosity of the device.

Antibodies or nucleic acids may be complexed or otherwise associated with other excipients contained in the microparticle composition that alter or enhance the biological effect, biological activity, stability, or release of the antibody or nucleic acid. In another aspect of the present invention, these agents may simply be incorporated into the microparticle composition along with the antibody or nucleic acid without otherwise forming a complex or association between the antibody or nucleic acid and the other agent. Antibody or nucleic acids in the form of prodrugs (including polymeric prodrugs) may be incorporated into the microparticle compositions of the present invention. Further aspects of the present invention include the incorporation of antibody or nucleic acids that have been otherwise chemically modified (for example, for purposes of achieving biological targeting or for other means of affecting the pharmacokinetics or biodistribution of the native antibody or nucleic acid or any combinations of the above.)

### Other Components

Other components such as, for example, solvents, suspension agents, surfactants, carriers, diluents, fillers, etc. that are typically used for the delivery of a free antibody or nucleic acid and for the delivery of long-acting formulations can also be used herein. One of skill in the art would know how to select the proper carrier to deliver the free antibody or nucleic acid and the long-acting formulation. In various aspects, the carrier comprises water or is water.

In one specific aspect, the long-acting formulation can comprise an antibody dissolved in an aqueous solution and microencapsulated antibody suspended in the same aqueous solution. To effect release, the antibody is microencapsulated in 25- to 125-micron diameter microparticles containing a 85:15 poly(D,L-lactide-co-glycolide) excipient with an inherent viscosity of 0.7 dL/gm. The antibody content of the microparticles is less than 5 wt % so the microencapsulated antibody is not released until the microparticle excipient begins to substantially hydrolyze and resorb. Upon administration of the aqueous composition comprising dissolved antibody and microencapsulated antibody, the dissolved antibody first affords efficacy for 3 months. During this 3-month period, the microencapsulated antibody is not released and stays within the microparticles. However, after 3 months, the 85:15 poly(D,L-lactide-co-glycolide) excipient begins to substantially hydrolyze and resorb allowing for release of the microencapsulated antibody for the next 3 months.

Although several aspects of the present invention have been described in the detailed description, it should be understood that the invention is not limited to the aspects disclosed, but is capable of numerous rearrangements, modifications and substitutions without departing from the spirit of the invention as set forth and defined by the following claims.

### EXAMPLES

To further illustrate the principles of the present invention, the following examples are put forth so as to provide those of ordinary skill in the art with a complete disclosure and description of how the compositions, articles, devices, and methods claimed herein are made and evaluated. They are intended to be purely exemplary of the invention and are not intended to limit the scope of what the inventors regard as their invention. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### 1. PLG Microparticle Formulation of an Antibody (Prophetic Example).

An antibody is dispersed using a Polytron mixer into 2-mL of a solution of 10% 85:15 DL-PLG (inherent viscosity 0.4 dL/g) in methylene chloride at a loading level of 5% by weight of the antibody relative to the combined weight of drug and polymer. Mixing is performed at 2-4° C. for 15-20 seconds at which time the suspension is transferred into a 5-cc syringe. Using an 18-gauge needle, this suspension is then delivered to an in-line Silverson mixer at a rate of about 10 g/min at the same time that an aqueous solution consisting of 2 wt % poly(vinyl alcohol) (PVA) saturated with methylene chloride solvent. The PVA solution is delivered to the in-line mixer at a flow rate of about 50 g/min. The resulting emulsion is then immediately diluted with fresh distilled water delivered at a flow rate of about 250 g/min in an extraction coil placed downstream from the Silverson mixer. The effluent from the extraction coil is then transferred to a tank that is stirred at about 600 rpm. The total volume of effluent from the process is stirred for 2-4 hours to facilitate extraction of solvent from the suspension and to, thereby, harden the antibody-loaded polymer microparticles. After hardening, the microparticles are isolated by passing the effluent through 125-micron and 25-micron sieves. The product collected on the 25-micron is then washed with an excess volume of fresh, distilled water (for example, 4-6 L). The product is then dried at ambient pressure and temperature by placing the 25-micron sieve under a laminar flow hood for at least 12 hours. The product is then gently scraped off of the sieve and is stored desiccated and frozen.

For ocular administration, the antibody-loaded microparticles are combined with a 50 to 100-microliter solution of antibody. The mixture is then injected into the vitreous of the eye. Upon administration, the unencapsulated antibody has efficacy for 1 month or longer. During this time, the PLG microparticles release little or no antibody. After the unencapsulated antibody is gone or no longer efficacious, the microencapsulated antibody begins to release. This release of microencapsulated antibody can be designed to occur for days, weeks of months. The release of microencapsulated antibody is therefore delayed until the unencapsulated antibody is no longer efficacious. The use of unencapsulated antibody means less microencapsulation polymer is needed and therefore more bioactive can be administered in the designated 50 to 100-uL volume.

Various modifications and variations can be made to the compositions, articles, devices, and methods described herein. Other aspects of the compositions, articles, devices, and methods described herein will be apparent from consideration of the specification and practice of the compositions, articles, devices, and/or methods disclosed herein. It is intended that the specification and examples be considered as exemplary.

What is claimed is:

1. A method of extending the release profile of an antibody or nucleic acid in a subject while reducing the total system mass of the polymer material of a biodegradable, long-acting formulation comprising administering to the subject at about the same time a free antibody or nucleic acid and a biodegradable, long-acting formulation containing the antibody or nucleic acid, wherein the free antibody or nucleic acid has a pharmaceutically acceptable bioactivity period of at least a week and wherein the biodegradable, long-acting formulation releases its antibody or nucleic acid to coincide with the diminution of activity of the free antibody or nucleic acid.

2. The method of claim 1, wherein the free antibody or nucleic acid and the long acting formulation are part of one unitary formulation.

3. The method of claim 1, wherein the administration is to a local delivery site.

4. The method of claim 1, wherein the administration is an ocular administration.

5. The method of claim 1, wherein the administration is an interarticular administration.

6. The method of claim 1, wherein the administration is to the central nervous system.

7. The method of claim 1, wherein the administration is to a tumor.

8. The method of claim 1, wherein the administration is an intradermal administration.

9. The method of claim 1, wherein the free antibody or nucleic acid has a pharmaceutically acceptable bioactivity period of at least two weeks.

10. The method of claim 1, wherein the free antibody or nucleic acid has a pharmaceutically acceptable bioactivity period of at least three weeks.

11. The method of claim 1, wherein the free antibody or nucleic acid has a pharmaceutically acceptable bioactivity period of at least four weeks.

12. The method of claim 1, wherein the biodegradable, long-acting formulation comprises a microparticle.

13. The method of claim 1, wherein the biodegradable, long-acting formulation comprises an implant.

14. The method of claim 1, wherein the antibody or nucleic acid comprises an antibody that specifically binds tumor necrosis factor-alpha (TNFα), vascular endothelial growth factor-A (VEGF-A), CD20, α4-integrin, or beta-amyloid.

15. The method of claim 1, wherein the antibody or nucleic acid comprises a small interfering RNA (siRNA) or antisense oligonucleotide.

16. The method of claim 1, wherein the subject is a human.

17. A controlled release formulation comprising a free antibody or nucleic acid and a biodegradable, long-acting formulation containing the antibody or nucleic acid, wherein the free antibody or nucleic acid has a pharmaceutically acceptable bioactivity period of at least a week and wherein the biodegradable, long acting formulation releases its antibody or nucleic acid to coincide with the diminution of activity of the free antibody or nucleic acid.

18. The controlled release formulation of claim 17, wherein the free antibody or nucleic acid has a pharmaceutically acceptable bioactivity period of at least two weeks.

19. The controlled release formulation of claim 17, wherein the free antibody or nucleic acid has a pharmaceutically acceptable bioactivity period of at least three weeks.

20. The controlled release formulation of claim 17, wherein the free antibody or nucleic acid has a pharmaceutically acceptable bioactivity period of at least four weeks.

21. The controlled release formulation of claim 17, wherein the biodegradable, long-acting formulation comprises a microparticle.

22. The controlled release formulation of claim 17, wherein the biodegradable, long-acting formulation comprises an implant.

23. The controlled release formulation of claim 17, wherein the free antibody or nucleic acid and the long acting formulation are part of one unitary formulation.

24. The controlled release formulation of claim 17, wherein (1) the free antibody or nucleic acid and (2) the long acting formulation are separately contained in a kit.

25. The controlled release formulation of claim 17, wherein the antibody or nucleic acid comprises an antibody that specifically binds tumor necrosis factor-alpha (TNFα), vascular endothelial growth factor-A (VEGF-A), CD20, α4-integrin, or beta-amyloid.

26. The controlled release formulation of claim 17, wherein the antibody or nucleic acid comprises a small interfering RNA (siRNA) or antisense oligonucleotide.
